(19) United States
(12) Patent Application Publication
(10) Pub. No.: US 2023/0035933 A1
(43) Pub. Date: Feb. 2, 2023

(54) System and Process For Managing Remote Services

(71) Applicant: Charles COLBY, Newbury Park, CA (US)

(72) Inventor: Charles COLBY, Newbury Park, CA (US)

(21) Appl. No.: 17/702,744

(22) Filed: Mar. 23, 2022

Related U.S. Application Data
(60) Provisional application No. 63/218,233, filed on Jul. 2, 2021.
(60) Provisional application No. 63/141,900, filed on Jan. 26, 2021.

(51) Int. Cl. G16H 80/00; G16H 40/67; H04L 65/1069; H04L 65/401

(52) U.S. Cl. CPC G16H 80/00; G16H 40/67; H04L 65/1069; H04L 65/4007

(57) ABSTRACT

A system and process for managing the delivery remote services to a succession of people, wherein a virtual waiting room is maintained, conditions of the waiting room are monitored, and new people are invited into the waiting room based on the monitored conditions. In the same or a different system, one or more remote service workstations are provided with a communications connection to a service provider, and a succession of persons use the workstation for receiving remote services while at least a substantial part of the communications connection is maintained between successive sessions. The system and process are particularly useful for remote medical services.

### RELATED APPLICATIONS

The present application claims benefit of U.S. provisional application 63/141900 filed Jan. 26, 2021, and application 63/218233 filed Jul. 2, 2021, the disclosures of which are incorporated herein by reference.

### BACKGROUND OF THE INVENTION

The invention relates to a system and process for managing remote physician services and observations to groups, large and small, to enable dynamically increased patient throughput and optimize efficiency

This invention is specifically designed for medical professional provided services from one or more remote locations, and is particularly suitable for the provision of services in connection with for self-collected samples, and/or services to large groups to dramatically improve productivity.

While physician observations, televisits, and remote observation of physician assisted testing are not new, the existing flow methods and practices are very limited when it comes to the number of patients that can be serviced in an hour. Prior systems require an individual connection to patients to be initiated with each patient. The amount of time per patient visit has been in the range of 5-10 minutes, with the throughput then being in the range of 6-12 per hour. There is a need, then, for an improved system and method for handling remote medical visits and consultations with improved patient throughput.

In some implementations of a remote medical services system, patients from respective locations (e.g., from their homes) will connect to the medical system in advance of an actual session with the medical professional, and will wait in a queue until the medical professional is available. If there are a large number of people to be serviced, the wait can be quite long. A common approach is to have every caller enter into a large queue and deliver to them a waiting message, e.g., you are number “XXX” in line and your expected wait time is “YYY”. This results in an inefficient user experience and a poor ability to manage available customer service resources and poor customer experience. A particular problem is that if you simply open a waiting list/queue or virtual room waiting room, you never know how many people you are going to get into the room at one specific time, and thus either have too many people in the room to be able to efficiently manage them, resulting in exceptionally long waiting times and poor customer support, or too many staff assigned into that virtual room such that you are inefficient in your costs associated with dealing with these people.

A possible partial solution is to limit how many people can make an appointment in a given time slot, but the benefits of this are illusory. For a given staffing, reducing the number of appointments leads to reduced waiting times, but does so at the cost of possible inefficient use of staffing resources if people do not show up for scheduled appointments or if appointments go faster than expected, and further reduction in patient throughput if appointment times go unused. Maximizing throughput can be addressed by overbooking appointments, but this leads back to the risk that all will show up causing a backlog and inappropriate wait times.

Another alternative solution is to simply pick an arbitrary number of people (e.g., 10), reach out to all of them, and once they have been cycled thru then invite another 10 people. While this addresses some problems, it is still wholly inefficient and requires substantial intervention on the part of administrative personnel or on the part of the service provider(s), who in the example described herein are qualified medical professionals. In the specific case of a healthcare application where the customers are patients and there are multiple physicians who need to interact with a list of patients, there may be a need to deliver medical information to a large number of patients and only a finite amount of resources to do this with. Complicating the issue is that the users may be disenfranchised from the provider and are being serviced over the Internet, and it is possible that when the provider decides to reach out to them they may not be available. So it isn't realistic to simply reach out to “XXX” number of people in a group at a time and then once that group is finished reach out to others. This is a half measure but not efficient and requires a tremendous amount of intervention. Physician resources are both expensive and limited in their availability. So there is a need to maximize the efficiency of their working hours by limiting their “down time” wasted between interactions with patients.

Yet another solution is to allow customers the option of having the system call them back “holding their virtual place in line”. However, this suffers from issues of the system properly connecting the people and or making the connection only to have the call drop and then the system doesn't try to reconnect with them. If the customer leaves a call-back number but the call-back fails, the system may “think” it has completed its mission, but the customer never got through. Or the customer may have called in again and resolved his or her issues, but the system doesn't know this and again contacts the customer based on the earlier call-back request. Again, inefficient and annoying to the consumer as well as to call service agents being connected to customers who are not on the line.

Thus, previous attempts to create and maintain a virtual queuing environment fail to address the overall problem of having an indeterminate number of people needing to interact with customer service providers (medical or otherwise) and typically limited resources and available staff to interact with those customers efficiently, leading to poor customer experience and/or poor management of resources. This results in frustration on the part of customer service agents or staff, and increased company costs.

### SUMMARY OF THE INVENTION

According to a first aspect of the invention designed to maximize patient throughput, there is at least one first terminal where a service (e.g., medical) professional is located, at least one second terminal at a location or locations remote from the first terminal, a televisit connection maintained between a first and at least one of said second terminals, and a succession of patients use each second terminal for a televisit with the medical professional, without having to re-establish for each patient at least the time-consuming part of the connection between the first and second terminals. This results in a very substantial increase in throughput, in some cases a ten-fold increase through each workstation. There can be multiple workstations and rooms set up simultaneously if desired, to further increase patient throughput.

According to a further aspect of the invention, there is at least one first terminal where a service (e.g., medical) professional is located, an electronic queue of patients awaiting a video and/or audio connection to said medical professional at said first terminal, and a queue management component that dynamically manages the queue by monitoring queue attributes such as the capacity of the queue, the number of service professionals servicing the queue, the average patient wait time in the queue, the minimum, maximum or average amount of time taken for each patient televisit, the minimum, maximum or average amount of time that elapses from the time a patient is invited into the queue until that patient is ready for a televisit with the service professional, and/or a likelihood that a given patient will join the queue when invited to do so, and invites patients into the queue in accordance with the monitored queue attributes.

Thus, this second aspect of the invention is directed to a dynamically adjusting virtual waiting room application that intelligently load balances and manages groups of people who need to interact with or serve others. In the example described and illustrated herein, the waiting room is adapted to apply to the health care field where a group of physicians need to interact with a group of patients via the web; however, the invention can be used across any service entity in which a group of people need to interact with others and efficiency of interaction resources is both beneficial and more cost effective.

The system also matches patients with physician resources based on the applicable licensed areas of physician practice in medicine, ensuring, by way of example, that California patients are connected to only California licensed medical professionals. While this is applicable in business cases in which specific state licensure are required for staff to interact with its patients/clients, this ability can be used in other general business uses; e.g., matching English speaking clients with English speaking staff and Spanish speaking clients with Spanish language speaking staff, etc. This is determined by the client/patient profile settings dynamically and routed by the system.

### BRIEF DESCRIPTION OF THE DRAWINGS

The invention will be more clearly understood from the following detailed description of non-limiting examples of the invention, in conjunction with the accompanying drawings wherein:

FIG. 1 is an illustration of a conference room implementation of the invention;

FIG. 2 is an illustration of an implementation of the invention using pop-up tents;

FIG. 3 is an illustration of the invention in a facility where patients cannot be brought to the static televisit connection; and

FIGS. 4 and 5 are diagrams of the system architecture and function according to a second aspect of the invention, showing a waiting room queue and workflow diagram for use of the invention in a healthcare application.

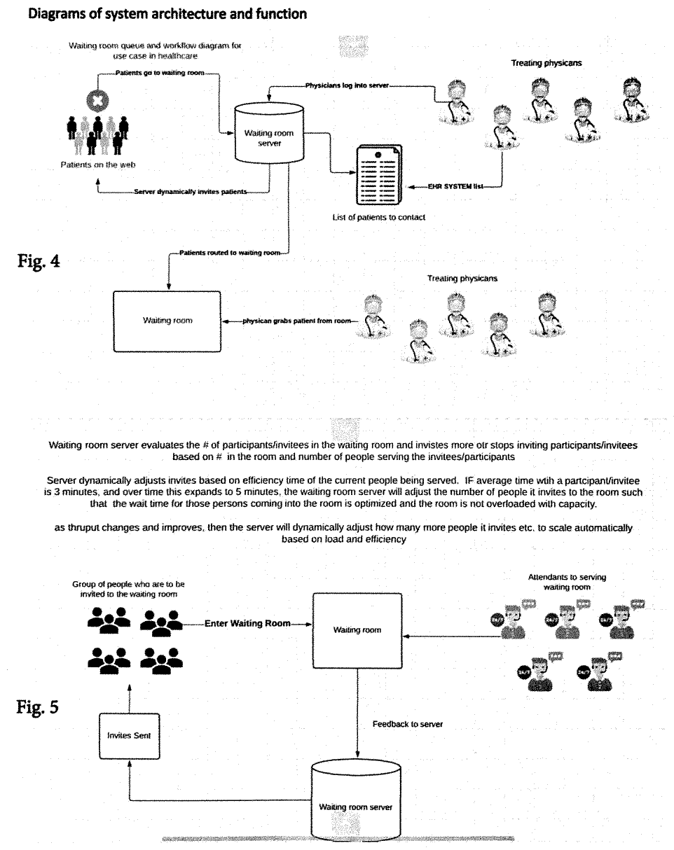

### DETAILED DESCRIPTION OF THE INVENTION

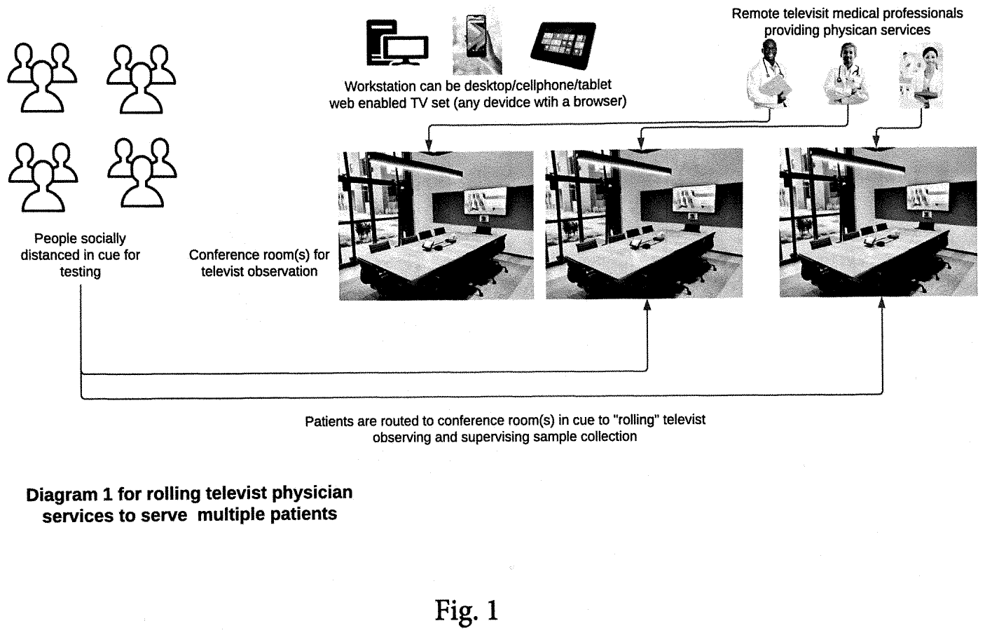

FIG. 1 is a simple illustration of a conference room implementation of the invention, wherein one or more conference rooms 10 are each equipped with a workstation 12 in communication with a service provider (e.g., physician) 14 at a different location. Patients wait in a queue for each conference room, or one or more queues can be shared by multiple conference rooms. When a patient completes a televisit and departs, a next patient can be admitted to the room for the next televisit. The connection between the physician and the workstation in any given conference room can be constant, so that the next televisit can commence promptly on patient entrance into the conference room, without having to wait for a connection to be established, resulting in significant throughput.

Alternatives still within the scope of the invention would include having only a part of the connection between physician and conference room workstation be continuous, so that the connection could be completed quickly and easily to commence the televisit. For example, a single physician could serve two conference rooms from a physician terminal, selecting a connection to a first conference room to conduct a televisit, and on completion of that first televisit selectively connecting to the second conference room where a new patient will be waiting, and while that second televisit is underway a third patient can be admitted to the first conference room to await a connection to the physician. When the physician connects to the conference room, the physician can also be presented with relevant medical or personal information about the patient, which can be facilitated by associating with each patient a QR code while the patient in in the queue or earlier, and having that QR code scanned on entry into the conference room.

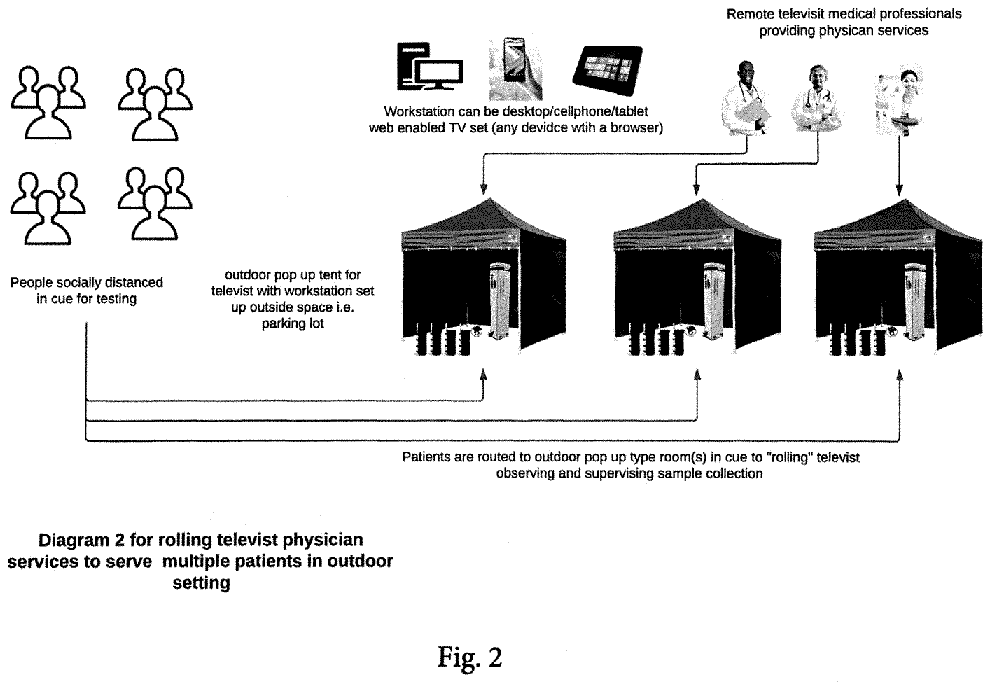

FIG. 2 is an illustration similar to FIG. 1 except that the patient televisit facilities are temporary structures, e.g., tents, set up in a parking lot instead of indoor conference rooms.

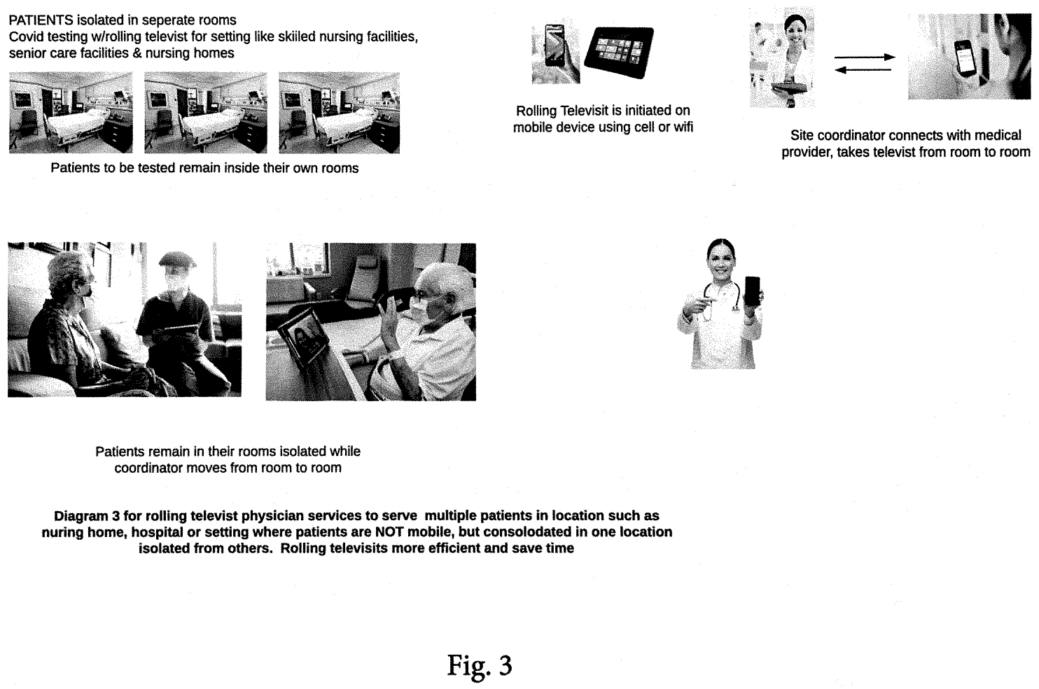

FIG. 3 illustrates an alternative where the patient televisit facility is even more portable than in FIG. 2. In this scenario, the patient televisit facility is a laptop or other portable device that can be brought to the patient instead of requiring the patient to come to the terminal, particularly useful in hospitals, nursing homes or other communities where there are televisit patients who are less mobile.

The following is a description of a non-limiting example of a manner in which the invention may be used.

### The Work Flow—

1) Patients are optionally first registered in a patient tracking system, which could be any patient tracking system but could be one such as described in U.S. Provisional Application 63/086050 entitled Patient, Information, Tracking and Evaluation System and Method, filed Oct. 1, 2020, the disclosure of which is incorporated herein by reference. Necessary typical steps can then be taken to be prepared to collect a sample for physician assisted testing.
2) Once a patient is qualified for testing, the patient is then placed in the “queue” to see the physician to assist testing observation and supervision.
3) The site administrator sets up a televisit session between a medical professional and a terminal, which may be in a conference room as illustrated in FIG. 1. Though the invention can be practiced with a single conference room, patient throughput needs may warrant multiple televisit rooms as shown in FIG. 1. Alternatively, there can be multiple workstations in a single conference room, separated from one another sufficiently to promote individual privacy between medical professionals and the patients in their interactions. The connection could be via any web enabled device (any device that has a browser that is capable of internet transmission and/or wifi) inside each conference room.
4) A patient from the cue is led into a conference room for observation and physician assisted testing.
5) At the outset of the televisit, the medical professionals identify patients and authenticate them via their birthdate and optionally other information given by the patient over the communications connection, comparing that to the information from the registration in the system, and the physician services are then performed. Such services include evaluation and management services (E&M visits), staff or medical professional observation of physician assisted testing, and sample collection, consultations, general medical professional services.

While it is contemplated that web-enabled devices be used for the televisit connections, in some circumstances (e.g., a medical facility) where there are medical professionals on site, it might be that the televisit could be conducted via wired connection such as a LAN.

While FIG. 1 illustrates the invention implemented via one or more conference rooms, it can be implemented with any form of televisit “room,” e.g., a warehouse with multiple workstations could serve the function of multiple conference rooms. Basically, any location where a work station can be set up.

The invention could also be implemented using temporary facilities, e.g., FIG. 2 showing one or more outdoor pop-up tents in place of multiple conference rooms, each tent having the equipment needed for a televisit, which can be a computer with a large screen, or could be a smaller tablet, or even a smartphone. Each pop-up tent could serve a respective cue, or all pop-up tents could serve a single cue.

In FIGS. 1 and 2, the televisit workstation is stationary and the patients wait in a cue to be brought to the televisit work station. It may instead be the case that the televisit workstation is mobile, and it is carried to each patient in succession. An example may be as illustrated in FIG. 3, where the patients are in hospital rooms or other rooms in a nursing home. Or there could be a mixed system/workflow where patients wait in a physical cue and successively enter the televisit area, but for some less mobile patients the televisit is brought to them.

In a yet further implementation, not shown, the patients may wait in the “queue” by sitting in their cars, with a staff person carrying a tablet from one car to the next to perform successive televisits.

In yet another example, all patients may be in line in their cars, with the televisit workstation being a drive-up window/monitor.

Thus, these various implementations may be viewed as successive levels of portability, e.g., the conference room implementation of FIG. 1 where the workstation is fixed in a fixed conference room, the pop-up tent configuration of FIG. 2 where the workstation is fixed in a temporary room (pop-up tent), and the mobile workstation configuration of FIG. 3 where neither the room nor the workstation are fixed, but the workstation is brought to the patient.

Finally, while there may be one medical professional for each established televisit connection, it would also be possible to have one medical professional handle multiple televisit connections. This could be done in a variety of ways, e.g., a physician could be in a room with multiple terminals each maintaining a constant connection with a respective televisit location (e.g., a conference room, pop-up tent, etc.), or the physician could be in a room with only a single terminal maintaining constant televisit connections with multiple locations, and the ability to rapidly switch back and forth between them. What is important is that there be some sort of constant connection maintained with each location so that it need not be set up again for each patient.

Single or multiple locations can be operated at the same time. I.e. , for a corporation with offices in 4 or 5 cities that has multiple employees, these locations and venues can be set up at the same time and patients/employees or people to be tested can be routed through the observation areas simultaneously.

The system also matches patients with physician resources based on the applicable licensed areas of physician practice in medicine, ensuring, by way of example, that California patients are connected to only California licensed medical professionals. While this is applicable in business cases in which specific state licensure are required for staff to interact with its patients/clients, this ability can be used in other general business uses; e.g. matching English speaking clients with English speaking staff and Spanish speaking clients with Spanish language speaking staff. This is determined by the client/patient profile settings dynamically and routed by the system.

Medical professionals can be any level of qualification depending upon the service and/or requirement for the service being rendered via the system of this invention. Any thus qualified staff based on regulations can manage the observation service commensurate with their qualifications for providing such services under AMA guidelines. See FIGS. 1 and 2.

Note that this system is mimicked in the virtual space in the case where remote users are self collecting their sample vials. Patients are routed into a virtual waiting room(s) where they are assigned to a separate one-on-one teleconference with physician/medical staff/medical professional for sample observation and sample collection. The process is similar to the above workflow, however it is done virtually not in a physical work setting.

With the inventive system and method, there is a first terminal where a medical professional is located, a second terminal at a location remote from the first terminal, a televisit connection maintained between the first and second terminals, and a succession of patients use the second terminal for a televisit with a medical professional, without having to re-establish for each patient at least the time-consuming part of the connection between the first and second terminals. This results in a very substantial increase in throughput, in some cases as much as 50-60 patients per hour through each workstation. There can be multiple workstations and rooms set up simultaneously if desired to increase patient throughput. This enables us to dramatically increase throughput and treat up to 10 times more patients per hour and facilitate the sample collection.

Rather than have the televisit patients conduct televisits from dedicated terminals as discussed above, they can each connect to the service provider from their own terminals, e.g., home computers or tablets, but this raises a concern about patient throughput while waiting for connections to be established, as discussed above. This can be addressed by having each service provider (e.g., physician) able to switch back and forth quickly between two sessions, with the next patient establishing his/her connection with one session during the time that the first session is ongoing. To ensure that the next connection is established before the current session is completed, patients can be queued up in a “virtual” waiting room. In this case, the service provider (e.g., physician) can log into a server from the web on any device. Once they have authenticated themselves into the system, and initiate that they are ready to start disseminating information, the system will do the following:

It will look at the total number of people who are providing the information (e.g., physicians) and calculate the number of people to “invite to interact” or join the waiting room to interact with the physicians. E.g., the system dynamically keeps track of the basic interactions and the time taken from the time the physician connects with the patient and the time the physician terminates the interaction or call. This is a number the system monitors and adjusts itself based on the dynamics of this time/number. If the average time to deal with a patient becomes greater, the system will slow down the number of invites into the waiting queue. If the interaction time average goes down, then the system will use that information and increase marginally the number of people it invites into the room. This number ‘time to manage an appointment’ can also be hard coded by default in the system administration panel by the administrator; i.e., make each appointment a default time of 3 minutes. The appointment time is constantly kept up and monitored such that the system knows what the expected throughput time is based on historical information (and can adapt to any workflow or business case accordingly). This in turn interacts with the system as described above to rate limit or increase the inflow of people into the queue.

The system will then analyze and update on a constant basis how many physicians (and/or other service providers) are online and signed into the waiting room server, and will monitor other visit-related data such as the average time required for each televisit, and will use this information to adapt its invitations accordingly. For example, the system will dynamically and automatically adjust its criteria for inviting patients into the virtual waiting room based at least in part on the ‘average time’ each appointment takes and on the dynamic number of physicians available to manage the visits. Additional relevant data may be the percentage of people who actually join the virtual waiting room once invited, and the amount of time it takes from them to join. By way of example only, the system may process this televisit-related data and determine a minimum number of patients to be maintained in the waiting room in order to ensure that there is always a patient online and ready when a physician wants to begin a next session, and then automatically invite new patients into the waiting room on a rolling basis to maintain a target waiting room population. A suitable target would be to keep a minimum number of people/patients in the waiting room queue needed to ensure efficiency of the service providers (physicians) while also not exceeding some maximum waiting room population that would result in waiting times that would be unnecessarily long. The appropriate minimum and maximum numbers would be dynamically updated. It is to be noted that a simple algorithm would be to look at the most recent patient to begin a televisit, see how long that patient had to wait, and to then increase or decrease the invitation rate based on this number. But the use of this number alone in the invitation control feedback loop would have an excessive latency, so in the preferred embodiment other factors are monitored to enable a meaningful prediction of how long the wait is likely to be for each patient newly admitted to the queue.

The system tracks ‘open invites’ (those who have been invited but have not yet joined) and the number of people in the waiting room at one time. It balances this against its aforementioned heuristics on appropriate load levels and as well measures and tracks the average time to manage an interaction between the doctors in our case, and the patients. In a business setting it could be customer service employees and customers to be assisted. The system then keeps track of these statistics and automatically invites more people into the virtual waiting room to keep the queue load (people in the waiting room) above the calculated minimum. If the waiting room population reaches or exceeds the maximum, e.g., dues to longer than expected televisit times and/or a higher than expected percentage of invitees joining the waiting room, the system will stop inviting people to join, until the number in the waiting room queue drops below the calculated maximum.

Instead of just halting invitations at some prescribed maximum waiting room population, the overpopulation concern could be addressed in multiple stages. E.g., when the waiting room population exceeds a first level, invitations to join the waiting room could still be issued, but anyone subsequently joining the waiting room could be presented with a message advising them that wait times may be longer than normal. If the waiting room population continues to rise and exceeds a second level, invitations to join the waiting room could be stopped, but people joining the waiting room in response to previous invitations would be presented with the extended wait time message. It would be possible to deny entry to the waiting room to anyone trying to join after the number of people in the waiting room exceeds a third level, although this would probably not be necessary if the person joining is presented with the extended wait time message and still wishes to join, and if that joining does not slow down the response to anyone ahead of them in the waiting room queue.

The system dashboard interface keeps track and displays those users who have not responded to the invitations, and will send a pre-configured number of re-invitations to them if unresponsive (the system default is 3) and then stop sending invites that haven't responded yet. However, these individuals will be in a table/display such that an operator can reach out if desired by a Voip phone call, to make a connection with the non-responsive invitations.

If the system is set up to deny entry to the waiting room, e.g., if the current wait time would be above a pre-set number (i.e. 30 minutes) the system could deny entry to the waiting room and explain to that individual that the room is full at this time and they will have to try their invitation later. When the number of people in the waiting room drops to a sufficient level, that individual could be notified that now the invitation is now valid and they should then re-join the room.

This invention can be used as well to route people dynamically or load balance customer service portals directing inbound traffic to the most efficient server at the time.

With this system we always have an efficient and dynamic number of people in the waiting room at all times such that the doctor's time is used efficiently and the patients are not waiting an excessive amount of time. This can be applied across many business cases and circumstances where virtual load management of appointment queue's is important for end user experience.

The system can also display statistics to the doctors in the room as to how many invitations are outstanding and how many have been accepted. Dynamically augmenting the numbers and invitations based on conditions of number of physicians (or people) who are able to interact with the public or list of people whom they need to interact with. As more physicians are able to sign onto the waiting room, the system adapts by sending out more invitations to the waiting room.

The function of the virtual waiting room itself is to ensure that the people addressing or interacting with the public are moving efficiently from one interaction to the next with as little down time between them as possible to maximize our resources efficiently.

### Audio/Video Preparation—

Another feature of the waiting room according to the present invention is that the system (e.g., in the circumstance when each visit is a video chat) will verify that both the audio and video of the patient are functioning properly prior to allowing them into the room. In this fashion, the system can ensure that all people in the room are able to correctly communicate with the person (e.g., physician) who is servicing the room. The system does not let a person into the waiting room unless the person's audio and/or video capabilities have been verified such that when the doctor picks up the call from the waiting room queue, that person has already verified that their device is functioning properly so that there is no technical delay in the connection or interaction itself. This improves efficiency and throughput of limited medical professional resources to manage customers/patients resulting in less cost, more efficiency and better customer experience/less waiting and dead time.

While the concept of a virtual waiting room or virtual queue is not new itself at all and has been used in some form or another for a while, the concept and invention of how to manage that queue is completely unique in its process and approach. Specifically, how people are brought into the virtual queue/waiting room. An advantage of the invention is that, while described herein in the context of delivering patient results from laboratory tests, the system and method of the invention can be used for any type of customer service circumstance where you have to manage a known group of people (patients in the described example) and efficiently interact with them in some manner in real time in any medium; audio, video, audio/video, text sms, web-based chat, etc.

This aspect of the invention is a “virtual” waiting room that managed by an application that may run on a remote and/or local server. The software is ubiquitous and platform agnostic to manage a list of people via synchronous or asynchronous communications (either video, audio, both or simply even sms/chat communication) and runs on a server using very little resources. The system monitors the status of the waiting room (how many people are in the room currently) and automatically invites additional users into the waiting room to ensure that the waiting room is never empty but at the same time keeping the wait time in the waiting room as low as is practical. Hence the system dynamically manages without intervention from an administrator the waiting queue to optimize the efficiency balance between incoming patients and time to treat them and address their concerns.

This system maintains HIPAA compliance when used in a medical setting, and can be easily integrated into any EHR/CMS system for any business or education application in which a single individual or group of individuals needs to interact with and exchange communications with another larger group of people; connected via the web. The application can work with ANY EHR or existing system that is capable of outputting a .csv (comma, separated value file) with only name and cell phone number. The invention therefore is not limited in its scope of use for a particular type of business or use. In our own use we built the system for our telemedicine practice. But it could easily be used for any other industry.

This system can be easily integrated into any EHR/CMS system for any business or education application in which a single individual or group of individuals needs to interact with and exchange communications with another larger group of people; connected via the web. While the invention is specifically helpful in telemedicine, a significant advantage of the invention is that it can be used for any type of customer service dilemma where you have to manage a known group of people (patients in our case) and efficiently interact with them in some manner in real time in any medium; audio, video, audio/video, text sms, web based chat etc.

While the concept of a virtual waiting room or virtual queue is not itself new and has been used in some form or another for some time, the concept and invention of how the queue is managed is novel in its process and approach. Specifically how and when persons are brought into the virtual queue/waiting room, and ensuring that their systems are functioning properly (audio and video capabilities as applicable).

1. A method of managing interaction between a plurality of users and at least one provider, the method comprising the following steps controlled by a computer:
maintaining a virtual waiting room from which users are selected for interaction with said at least one provider;
monitoring at least one changing characteristic of said virtual waiting room;
inviting additional users into said virtual waiting room based at least in part on the monitored characteristic.

2. The method according to claim 1, wherein said at least one characteristic comprises a predicted time from entry of a user into said waiting room until beginning interaction with said provider.

3. The method according to claim 1, wherein said at least one characteristic comprises a characteristic that affects the length of time from entry of a user into said waiting room until beginning interaction with said provider.

4. The method of claims 3, wherein said at least one characteristic comprises a number of users in said virtual waiting room.

5. The method according to claim 3, wherein said at least one characteristic comprises a number of providers servicing said virtual waiting room.

6. The method according to claim 3, wherein said at least one characteristic comprises an average time required to service each user once the user begins interacting with the service provider.

7. The method according to claim 1, wherein users in said virtual waiting room are serviced by a plurality of service providers, and wherein said method further comprises the step of connecting a particular user in said waiting room to a particular one of said plurality of providers based on at least one characteristic of said particular user and at least one complementary characteristic of said particular provider.

8. The method according to claim 7, wherein said at least one characteristic is a geographical location of said particular user.

9. The method according to claim 7, wherein said at least one characteristic is a language spoken by said particular user.

10. The method according to claim 7, wherein there are a plurality of said waiting rooms and said connecting step comprises connecting said particular user to said particular provider based on an attribute of the waiting room in which said particular user is waiting.

11. The method according to claim 1, wherein said provider is a healthcare professional.

12. A method of a medical service provider using a first terminal at a first location to provide a medical service to at least first and second patients, said method comprising the steps of:
providing a second terminal at at least one second location different from said first location;
said medical service provider providing said medical service to said first patient at said second location via a communications connection between said first and second terminals;
maintaining said communications connection while said second patient replaces said first patient at said second terminal; and
providing said service to said second patient via said communications connection.

13. The method according to claim 12, wherein said communications connection is an internet connection.

14. A method of a provider using a first terminal at a first location to provide a service to at least first and second users, said method comprising the steps of:
providing a second terminal at at least one second location different from said first location;
said provider providing said service to said first user at said second location via an internet communications connection between said first and second terminals;
maintaining said communications connection while said second user replaces said first user at said second terminal; and
providing said service to said second user via said communications connection.

15. The method according to claim 14, wherein said second terminal is fixed at said second location.

16. The method according to claim 14, wherein said second user is at a third location, and replacing said first user at said second terminal comprises moving said second terminal from said second location to said third location.

17. The method according to claim 14, wherein said provider provides said service to users at said second terminal and at a third terminal connected to said first terminal, and wherein said provider switches from said second terminal to said third terminal or from said third terminal to said second terminal to provide said service to a different user.

18. The method according to claim 14, wherein said service is medical consultation and said service provider is a healthcare professional.

19. The method according to claim 18, wherein said communications connection and said method are HIPAA compliant.

20. The method according to claim 14, wherein said provider authenticates said user based on information received over said communications connection.

21. A system for practicing the method of claim 1, said system comprising:
at least one terminal from which said provider can provide said service; and
at least one processor configured to maintain said virtual waiting room, monitor said at least one changing characteristic of said virtual waiting room, and control the inviting of additional users into said virtual waiting room based at least in part on the monitored characteristic.
